(19) United States
(12) Patent Application Publication
(10) Pub. No.: US 2009/0208959 A1
(43) Pub. Date: Aug. 20, 2009

(54) FLUORESCENCE-LABELED OLIGONUCLEOTIDE TO DETECT NUCLEIC ACID AND METHOD FOR ACQUIRING INFORMATION ABOUT DOUBLE-STRAND FORMATION BY USING FLUORESCENCE-LABELED OLIGONUCLEOTIDE TO DETECT NUCLEIC ACID

(76) Inventor: Takuya KISHIMOTO, Tokyo (JP)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/368,344

(22) Filed: Feb. 10, 2009

(30) Foreign Application Priority Data
Feb. 14, 2008 (JP) P2008-033164

(51) Int. Cl. C12Q 1/68; C07H 21/00

(52) U.S. Cl. 435/6; 536/24.3

(57) ABSTRACT

A fluorescence-labeled oligonucleotide to detect nucleic acid has a base sequence complementary to the nucleic acid chain to be detected and changes in fluorescence characteristics upon double-strand formation with the nucleic acid chain to be detected. The label is an intercalating fluorescent dye and a non-intercalating fluorescent dye. The intercalating fluorescent dye acquires excited energy upon absorption of light and gives it to the non-intercalating fluorescent dye, and the non-intercalating fluorescent dye receives the excited energy to emit light.

### CROSS REFERENCES TO RELATED APPLICATIONS

The present invention contains subject matter related to Japanese Patent Application JP 2008-033164 filed in the Japan Patent Office on Feb. 14, 2008, the entire contents of which being incorporated herein by reference.

### BACKGROUND OF THE INVENTION

1. Field of the Invention

The present invention relates to a fluorescence-labeled oligonucleotide to detect nucleic acid and a method for acquiring information about double-strand formation by using said fluorescence-labeled oligonucleotide to detect nucleic acid. More particularly, the present invention relates to a fluorescence-labeled oligonucleotide to detect nucleic acid which changes in fluorescence characteristics upon double-strand formation with a nucleic acid chain to be detected.

2. Description of the Related Art

For the purpose of elucidating the molecular mechanism of diseases, establishing the diagnosis of diseases, and searching the target for drug development, there have been proposed several techniques to quantitatively analyze the gene expression in cells or tissues.

The quantitative analysis of genes is accomplished mainly by optically detecting and determining the nucleic acid chain to be detected by using a probe and a variety of fluorescent dyes. The probe is an oligonucleotide having a base sequence complementary to the nucleic acid chain to be detected.

One example of such methods employs an intercalating fluorescent dye which emits fluorescence upon irradiation with excitation ray while it is bound or inserted between the base pair of double-strand nucleic acid.

Hybridization (to form double-strand) between the probe and the nucleic acid to be detected, which is performed in the presence of an intercalating fluorescent dye, causes the intercalating fluorescent dye to bind between the base pair of the resulting double-strand nucleic acid chain. By measuring the intensity of fluorescence emitted from the intercalating dye upon irradiation with excitation ray, one can determine the amount of double-strand formed and the amount of nucleic acid to be detected.

There is another method that employs TaqMan probe (registered trademark) which has the 5′ end modified with a fluorescent dye and the 3′ end modified with a quencher substance. This method is used for the real time PCR.

The TaqMan probe hybridizes specifically to the DNA to be detected in the annealing step of PCR. In this stage, the quencher is present in the probe and hence the fluorescent dye does not emit fluorescence upon irradiation with excitation ray. However, in the extension step, the probe which has hybridized to the DNA to be detected is decomposed by the Taq DNA polymerase (which has the 5′→3′ exonuclease activity). This decomposition librates the fluorescent dye from the probe and the fluorescent dye becomes free of the quencher and hence emits fluorescence. By detecting this fluorescence, one can determine in real time the amount of amplified PCR product and the amount of DNA to be detected.

Japanese Patent Laid-open No. 2006-296279 discloses a method for hybridization detection by means of an intercalating fluorescent dye. Japanese Patent Laid-open No. 2004-248676 discloses a method for quantitative detection of human β defensin 2mRNA by real time PCR that employs TaqMan probe.

### SUMMARY OF THE INVENTION

The related quantitative analysis of genes by means of the foregoing intercalating fluorescence dye or TaqMan probe suffers the disadvantaging of causing measurement errors due to variation in the amount of intercalating fluorescent dye or instability of the light source in the spectrofluorophotometer.

Such measurement errors can be avoided by preparing a calibration curve for each measurement which relates the concentrations of standard samples with the intensity of fluorescence detected.

However, preparing a calibration curve at each time of measurement is a very tedious work. Moreover, in the case of quantitative analysis of genes that employs multiplates, some wells are used for measurement of standard samples and this limits the number of samples that can be analyzed at one time and hence reduces the efficiency of analysis.

It is desirable to provide a fluorescence-labeled oligonucleotide to detect nucleic acid and a method for acquiring information about double-strand formation by using said fluorescence-labeled oligonucleotide to detect nucleic acid. According to an embodiment of the present invention, it is possible to carry out the quantitative analysis of genes simply and efficiently without the necessity of preparing calibration curves.

The present invention to tackle the above-mentioned problem is directed to a fluorescence-labeled oligonucleotide to detect nucleic acid which has a base sequence complementary to the nucleic acid chain to be detected and changes in fluorescence characteristics upon double-strand formation with the nucleic acid chain to be detected.

The fluorescence-labeled oligonucleotide to detect nucleic acid permits one to determine its ratio in the bound state or unbound state according to its fluorescent characteristics.

The fluorescence-labeled oligonucleotide to detect nucleic acid can be obtained by labeling with an intercalating fluorescent dye and a non-intercalating fluorescent dye.

Alternatively, it may be constructed such that a non-intercalating fluorescent dye emits light as it receives excited energy from an intercalating fluorescent dye which has absorbed light.

The present invention is directed also to a method for detecting information about fluorescence of the fluorescence-labeled oligonucleotide to detect nucleic acid by using the fluorescence-labeled oligonucleotide to detect nucleic acid, thereby acquiring information about double-strand formation between the fluorescence-labeled oligonucleotide to detect nucleic acid and the nucleic acid chain to be detected.

The method according to an embodiment of the present invention permits one to acquire information about the amount of nucleic acid chain to be detected because it permits one to measure the ratio of fluorescence-labeled oligonucleotide to detect nucleic acid in its bound state or unbound state.

The terms used herein are defined as follows. “Nucleic acid chain” includes single-strand and double-strand DNA and RNA.

“Intercalating fluorescent dye” denotes a fluorescent dye which emits fluorescence upon irradiation with excitation ray while it is bound to or inserted into the base pair of double-strand nucleic acid. Specifically, it includes SYBR Green, Pico Green (registered trademark), TOTO-1, POPO-1, and the like.

“Non-intercalating fluorescent dye” denotes any fluorescent dye which has no intercalating characteristics. It includes commonly used Cy3, Cy5, and Alexa Fluor (registered trademark).

The present invention provides a fluorescence-labeled oligonucleotide to detect nucleic acid and a method for acquiring information about double-strand formation by using said fluorescence-labeled oligonucleotide to detect nucleic acid. The present invention permits one to carry out the quantitative analysis of genes simply and efficiently without the necessity of preparing calibration curves.

### BRIEF DESCRIPTION OF THE DRAWINGS

FIGS. 1A and 1B are schematic diagrams showing the fluorescence-labeled oligonucleotide according to the first embodiment of the present invention;

FIGS. 2A and 2B are schematic diagrams showing the fluorescence-labeled oligonucleotide according to the second embodiment of the present invention;

FIGS. 3A and 3B are schematic diagrams showing the fluorescence-labeled oligonucleotide according to the second embodiment of the present invention, in which the intercalating fluorescent dye 1 is SYBR Green and the non-intercalating fluorescent dye 2 is Cy3; and

FIG. 4 is a diagram showing the fluorescence spectra obtained from standard samples.

### DETAILED DESCRIPTION OF THE PREFERRED EMBODIMENTS

The embodiments of the present invention will be described below with reference to the accompanying drawings. They are typical ones and are not intended to restrict the scope of the invention.

According to an embodiment of the present invention, the fluorescence-labeled oligonucleotide to detect nucleic acid (simply referred to as fluorescence-labeled oligonucleotide hereinafter) has a base sequence complementary to the nucleic acid chain to be detected and changes in fluorescence characteristics as it forms the double-strand with the nucleic acid chain to be detected.

“Change in fluorescence characteristics” means that the fluorescence which is emitted from the fluorescence-labeled oligonucleotide upon irradiation with excitation ray changes in wavelength after it binds (to form the double strand) to the nucleic acid chain to be detected. Here, it does not mean that any fluorescence-labeled oligonucleotide which does not emit fluorescence before binding with the nucleic acid chain to be detected changes into one which emits fluorescence after binding. In other words, the fluorescence-labeled oligonucleotide pertaining to an embodiment of the present invention is one which emits fluorescence in both states of binding and non-binding with the nucleic acid chain to be detected and which emits fluorescence varying in wavelength in each of the two states.

Since the fluorescence-labeled oligonucleotide pertaining to an embodiment of the present invention is constructed such that it changes in fluorescence characteristics as it forms the double-strand, it permits one to measure its ratio in the bound state and unbound state.

Moreover, the fluorescence-labeled oligonucleotide pertaining to an embodiment of the present invention permits one to determine the nucleic acid chain to be detected on the basis of its ratio in the bound state and unbound state.

The related way of detecting nucleic acid chain by means of intercalating fluorescent dyes or TaqMan probe resorts to detection of fluorescence resulting from the probe binding with the nucleic acid chain to be detected. This procedure prevents detection of probes in unbound state and hence prevents measurement of the ratio of probes in bound state and unbound state.

The procedure that employs an intercalating fluorescent dye and a probe is designed to detect fluorescence emitted from the intercalating fluorescent dye which has gets in between the base pair of the double-strand nucleic acid. Therefore, it cannot detect the probe in an unbound state. The procedure that employs TaqMan probe cannot detect the probe in an unbound state either, because fluorescence from the probe in an unbound state is inhibited by the quencher.

A related practice to eliminate this disadvantage is to relate the intensity of fluorescence resulting from the probe binding with nucleic acid chain to be detected with the amount of nucleic acid chain to be detected, thereby calculating the amount of DNA to be detected. For this purpose, it is necessary to previously prepare, by using standard samples with known concentrations, a calibration curve that relates the intensity of fluorescence with the concentration of samples.

In contrast to this related practice, the fluorescence-labeled oligonucleotide according to an embodiment of the present invention makes it possible to quantitatively determine the nucleic acid chain to be detected on the basis of the ratio of its bound state and unbound state, without the necessity of preparing a calibration curve.

The following is a detailed description (in reference to the accompanying drawings) of the constitution of the fluorescence-labeled oligonucleotide pertaining to an embodiment of the present invention and the method for acquiring information about the formation of double-strand with the help of the fluorescence-labeled oligonucleotide.

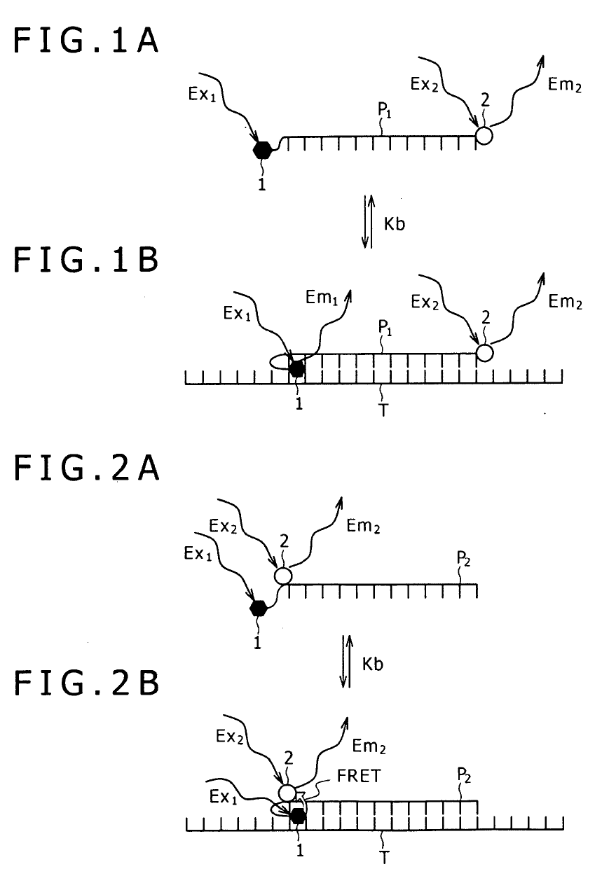

FIGS. 1A and 1B are schematic diagrams showing the fluorescence-labeled oligonucleotide according to the first embodiment of the present invention. FIG. 1A depicts the fluorescence-labeled oligonucleotide in its unbound state, and FIG. 1B depicts the fluorescence-labeled oligonucleotide in its bound state.

In FIGS. 1A and 1B, the symbol P₁ denotes a fluorescence-labeled oligonucleotide, which has the base sequence complimentary to the nucleic acid chain T to be detected (as shown in FIG. 1B) and is labeled with an intercalating fluorescent dye 1 and a non-intercalating fluorescent dye 2.

In FIGS. 1A and 1B, the symbol Ex₁ denotes the excitation ray corresponding to the excited wavelength of the intercalating fluorescent dye 1, and the symbol Ex₂ denotes the excitation ray corresponding to the excited wavelength of the non-intercalating fluorescent dye 2. Also, the symbol Kb denotes the binding constant (mentioned later) of the fluorescence-labeled oligonucleotide P₁.

When the fluorescence-labeled oligonucleotide P₁ in its unbound state (shown in FIG. 1A) is irradiated with the excitation rays Ex₁ and Ex₂, the non-intercalating fluorescent dye 2 emits the fluorescence Em₂ but the intercalating fluorescent dye 1 does not. That is, the intercalating fluorescent dye 1 emits fluorescence only when it is bound or inserted between the base pair of double-strand nucleic acid.

This means that the fluorescence-labeled oligonucleotide P₁ in its unbound state has the fluorescent characteristics that depend on the wavelength of the fluorescence Em₂.

By contrast, when the fluorescence-labeled oligonucleotide P₁ binds with the nucleic acid chain T (as shown in FIG. 1B), the intercalating fluorescent dye 1 binds or inserts between the double-strand formed by the fluorescence-labeled oligonucleotide P₁ and the nucleic acid chain T to be detected. Thus, when the fluorescence-labeled oligonucleotide P₁ is irradiated with excitation rays Ex₁ and Ex₂, not only the non-intercalating fluorescent dye 2 emits fluorescence Em₂ but also the intercalating fluorescent dye 1 emits fluorescence Em₁.

In other words, the fluorescent characteristics of the fluorescence-labeled oligonucleotide P₁ in its bound state depend on the wavelength of both fluorescence Em₁ and fluorescence Em₂.

As mentioned above, the fluorescence-labeled oligonucleotide P₁ varies in fluorescent characteristics (or emits Em₂ in its unbound state (as shown in FIG. 1A) and emits Em₁ and Em₂ in its bound state (as shown in FIG. 1B)). This makes it possible to detect the fluorescence-labeled oligonucleotide P₁ in its bound state with the help of fluorescence Em₁ and to detect the fluorescence-labeled oligonucleotide P₁ in its bound state and unbound state (or the total amount) with the help of fluorescence Em₂.

Therefore, if the sample is irradiated with excitation ray Ex₁ and excitation ray Ex₂ and the intensity I₁ of the resulting fluorescence Em₁ and the intensity I₂ of the resulting fluorescence Em₂ are detected and their ratio is determined, then it will be possible to know the ratio of the fluorescence-labeled oligonucleotide P₁ in its bound state to the total amount of the fluorescence-labeled oligonucleotide P₁.

For the ratio of the fluorescence-labeled oligonucleotide P₁ in its bound or unbound state to be measured, the fluorescent dye in the fluorescence-labeled oligonucleotide P₁ should exist in a fixed amount (in terms of the number of molecules). Otherwise, the intensity of fluorescence per molecule of fluorescence-labeled oligonucleotide P₁ varies, making it impossible to measure the ratio of fluorescence-labeled oligonucleotide P₁ according to the intensity of fluorescence.

The fluorescence-labeled oligonucleotide P₁ shown in FIGS. 1A and 1B has one molecule each of intercalating fluorescent dye 1 and non-intercalating fluorescent dye 2. It may have more than one molecule each of the fluorescent dye. However, the number of molecules of fluorescent dye for labeling should be fixed. For example, there may be three molecules of intercalating fluorescent dye 1 and two molecules of non-intercalating fluorescent dye 2.

The intercalating fluorescent dye 1 is not specifically restricted; it includes SYBR Green, Pico Green, TOTO-1, POPO-1, etc. which are in general use.

The non-intercalating fluorescent dye 2 is not specifically restricted; it includes Cy3, Cy5, and Alexa Fluor (registered trademark).

The fluorescence-labeled oligonucleotide P₁ shown in FIGS. 1A and 1B has one kind each of intercalating fluorescent dye 1 and non-intercalating fluorescent dye 2. It may have more than one kind each of the fluorescent dye. In this case, excitation rays with three or more wavelengths may be used to detect the fluorescence of each fluorescent dye.

Labeling with the intercalating fluorescent dye 1 and non-intercalating fluorescent dye 2 may be accomplished in the related way to prepare the fluorescence-labeled oligonucleotide P₁. These dyes may be attached directly to the 5′ end or 3′ end of the oligonucleotide. In the case of an oligonucleotide having thymine, either of the intercalating fluorescent dye 1 or the non-intercalating fluorescent dye 2 may be introduced into it with the help of thymine for inner fluorescence labeling. Incidentally, only thymine is available at present as the nucleotide for inner fluorescence labeling. However, any nucleotide for inner fluorescence labeling other than thymine may be used if available.

FIGS. 1A and 1B show instances in which the intercalating fluorescent dye 1 is attached to the 5′ end and the non-intercalating fluorescent dye 2 is attached to the 3′ end. However, the fluorescent dyes may be attached to any other positions.

The fluorescence-labeled oligonucleotide P₁ may have any length (or the number of bases) according to the base sequence of the nucleic acid chain T to be detected. Usually, the standard length is 15 to 30 bps. It is not always necessary that the base sequence be completely complementary to the nucleic acid chain T to be detected. There may be one or more mismatches (non-complementary base) so long as it can form the double-strand with the nucleic acid chain T to be detected at a desired temperature for hybridization.

FIGS. 2A and 2B are schematic diagrams showing the fluorescence-labeled oligonucleotide according to the second embodiment of the present invention. FIG. 2A depicts the fluorescence-labeled oligonucleotide in its unbound state, and FIG. 2B depicts the fluorescence-labeled oligonucleotide in its bound state.

In FIGS. 2A and 2B, the symbol P₂ denotes a fluorescence-labeled oligonucleotide, which, like the fluorescence-labeled oligonucleotide P₁ shown in FIGS. 1A and 1B, has the base sequence complimentary to the nucleic acid chain T to be detected and is labeled with an intercalating fluorescent dye 1 and a non-intercalating fluorescent dye 2.

The fluorescence-labeled oligonucleotide P₂ differs from the fluorescence-labeled oligonucleotide P₁ in that it has both the intercalating fluorescent dye 1 and the non-intercalating fluorescent dye 2 attached to the same end (or the 5′ end in FIGS. 2A and 2B). The intercalating fluorescent dye 1 absorbs light and gives excitation energy to the non-intercalating fluorescent dye 2, which in turn receives the excitation energy and emits fluorescence.

Transfer of excitation energy from the intercalating fluorescent dye 1 to the non-intercalating fluorescent dye 2 is generally referred to as “fluorescence resonance energy transfer” (FRET). FRET is a phenomenon that a fluorescent dye (as a donor) excites another fluorescent dye (as an acceptor) adjacent to it (within a distance of 1 to 10 nm) when it is excited. FRET occurs in the case where the emission spectrum of the donor overlaps the excitation spectrum of the acceptor, and the donor and the acceptor are close to each other with adequate orientation.

The fluorescence-labeled oligonucleotide P₂ has the intercalating fluorescent dye 1 as the donor (a fluorescent dye to be excited) and the non-intercalating fluorescent dye 2 as the acceptor (a fluorescent dye to be detected).

The intercalating fluorescent dye 1 has the emitting spectrum which overlaps the excited spectrum of the non-intercalating fluorescent dye 2. These two fluorescent dyes may be used in combination as shown in Table 1.

TABLE 1

| | Intercalating | Non-intercalating |
|---|---|---|
| | fluorescent dye 1 | fluorescent dye 2 |
| 1 | SYBR Green | TET |
| 2 | Pico Green | Cy3 |
| 3 | SYBR Green | Alexa532 |
| 4 | Pico Green | Alexa546 |

When the fluorescence-labeled oligonucleotide P₂ in its unbound state (shown in FIG. 2A) is irradiated with the excitation ray Ex₂, the non-intercalating fluorescent dye 2 emits the fluorescence Em₂ but the intercalating fluorescent dye 1 does not even though it is irradiated with the excitation ray Ex₁.

Consequently, the fluorescence-labeled oligonucleotide P₂ in its unbound state is characterized by that it emits fluorescence Em₂ upon irradiation with excitation ray Ex₂.

When the fluorescence-labeled oligonucleotide P₂ binding with the nucleic acid chain T to be detected as shown in FIG. 2B is irradiated with excitation ray Ex₁, the intercalating fluorescent dye 1 gets excited and the excited energy moves to the non-intercalating fluorescent dye 2 by FRET (indicated with a white arrow in FIG. 2B). As the result, the non-intercalating fluorescent dye 2 emits fluorescence Em₂.

In the case of irradiation with excitation ray Ex₂, the non-intercalating fluorescent dye 2 gets excited directly and emits fluorescence Em₂.

In other words, the fluorescence-labeled oligonucleotide P₂ in its bound state emits fluorescence Em2 upon irradiation with either excitation ray Ex₁ or excitation ray Ex₂.

As mentioned above, the fluorescence-labeled oligonucleotide P₂ varies in characteristics depending on its bound state. In its unbound state (as shown FIG. 2A), it emits fluorescence Em₂ upon irradiation with excitation ray Ex₂. However, in its bound state (as shown in FIG. 2B), it emits fluorescence Em₂ upon irradiation with excitation rays Ex₁ and Ex₂. Thus, fluorescence Em₂ arising from irradiation with excitation ray Ex₁ permits detection of the fluorescence-labeled oligonucleotide P₂ in its bound state, and fluorescence Em₂ arising from irradiation with excitation ray Ex₂ permits detection of all the fluorescence-labeled oligonucleotide P₂ in both its bound state and its unbound state.

Thus, if the intensity I₁ of fluorescence Em₂ arising from irradiation with excitation ray Ex₁ and the intensity I₂ of fluorescence Em₂ arising from irradiation with excitation ray Ex₂ are measured and their ratio is obtained, it is possible to know the ratio of the fluorescence-labeled oligonucleotide P₂ in its bound state to the total amount of the fluorescence-labeled oligonucleotide P₂.

The fluorescence-labeled oligonucleotide P₂ emits only one wavelength (Em₂) of fluorescence to be detected, unlike the fluorescence-labeled oligonucleotide P₁. This permits detection with a device of simple structure.

The fluorescence-labeled oligonucleotide P₂ is identical with the fluorescence-labeled oligonucleotide P₁ in that it should have a predetermined number of molecules of labeling fluorescent dyes, it should have two or more each of the labeling intercalating fluorescent dye 1 and the labeling non-intercalating fluorescent dye 2, and it can be applied to the base sequence of any length (with or without mismatch), as mentioned above.

Labeling for the fluorescence-labeled oligonucleotide P₂ with the intercalating fluorescent dye 1 and the non-intercalating fluorescent dye 2 may also be accomplished in the same way as for the fluorescence-labeled oligonucleotide P₁. However, in the case of the fluorescence-labeled oligonucleotide P₂, it is necessary that both the intercalating fluorescent dye 1 and the non-intercalating fluorescent dye 2 should be attached to the same end (or the 5′ end as shown in FIGS. 2A and 2B). The two fluorescent dyes should be close to each other within a distance of 1 to 10 nm, which is necessary for FRET to occur. This may be achieved by attaching the two dyes individually to two ends of a three-forked spacer and attaching the remaining end of the spacer to the 5′ or 3′ end through a reactive group. It is also possible to introduce either of the two fluorescent dyes into the fluorescence-labeled oligonucleotide P₂ with the help of thymine for inner fluorescence labeling. In this case, either of the two dyes which has been attached to the 5′ or 3′ end should be a certain distance (equivalent to one to five nucleotide units) away from the thymine which has been labeled with the other dye.

The following deals with the method for acquiring information about the amount of nucleic acid chain T to be detected by means of the fluorescence-labeled oligonucleotide P₁.

As mentioned above, the fluorescence-labeled oligonucleotide P₁ in its bound state can be detected with the help of fluorescence Em₁ and all of the fluorescence-labeled oligonucleotide P₁ in its bound state and unbound state can be detected with the help of fluorescence Em₂. Moreover, the ratio of the intensity I₁ of fluorescence Em₁ to the intensity I₂ of fluorescence Em₂ gives the ratio of the amount of fluorescence-labeled oligonucleotide P₁ in its bound state to the total amount of fluorescence-labeled oligonucleotide P₁.

Thus, the fluorescence-labeled oligonucleotide P₁ permits determination of the nucleic acid chain T to be detected by means of the following formulas.

Conc=Kb·(R−Ri)/(Ra−R) (1)

R=(I₁−B₁)/(I₂−B₂) (2)

In the formulas above, “Conc” denotes the concentration (in mol/L) of the nucleic acid chain T to be detected, and “Kb” denotes the binding constant of the fluorescence-labeled oligonucleotide.

“I₁” denotes the intensity of fluorescence Em₁ due to excitation ray Ex₁, and “B₁” denotes its background value (which is the intensity of fluorescence Em₁ emitted by irradiation with excitation ray Ex₁ in the absence of the fluorescence-labeled oligonucleotide P₁ in the reaction solution). “I₂” denotes the intensity of fluorescence Em₂ due to excitation ray Ex₂, and “B₂” denotes its background value.

“Ri” denotes the value of R which is measured in the absence of the nucleic acid chain T to be detected in the reaction solution. “Ra” denotes the value of R which is measured when the nucleic acid chain T to be detected is added in excess to the fluorescence-labeled oligonucleotide P₁. “Ra” and “Ri” should be previously measured by using the same measuring system.

The binding constant Kb can be calculated from the formula (3) below, in which “Tm” denotes the melting point calculated from the base sequence of the fluorescence-labeled oligonucleotide P₁, and “a” denotes a constant.

Kb=a·Tm (3)

The binding constant Kb may be previously obtained by using a standard sample of known concentration.

The method for assay by the fluorescence-labeled oligonucleotide P₁ merely needs to measure the intensity I₁ of fluorescence Em₁ and the intensity I₂ of fluorescence Em₂ and permits determination of concentration of nucleic acid to be detected from the thus measured intensity of fluorescence and the formulas (1) and (2) without the necessity of drawing calibration curves at each time of determination.

The advantage of the foregoing method is that the intensity I₁ of fluorescence is divided by the intensity I₂ of fluorescence (both calibrated by subtraction of background value) and this calculation cancels errors due to variation in amount of the intercalating fluorescent dyes and due to instability in light source in the spectrofluorophotometer. Therefore, the foregoing method is free of measurement errors.

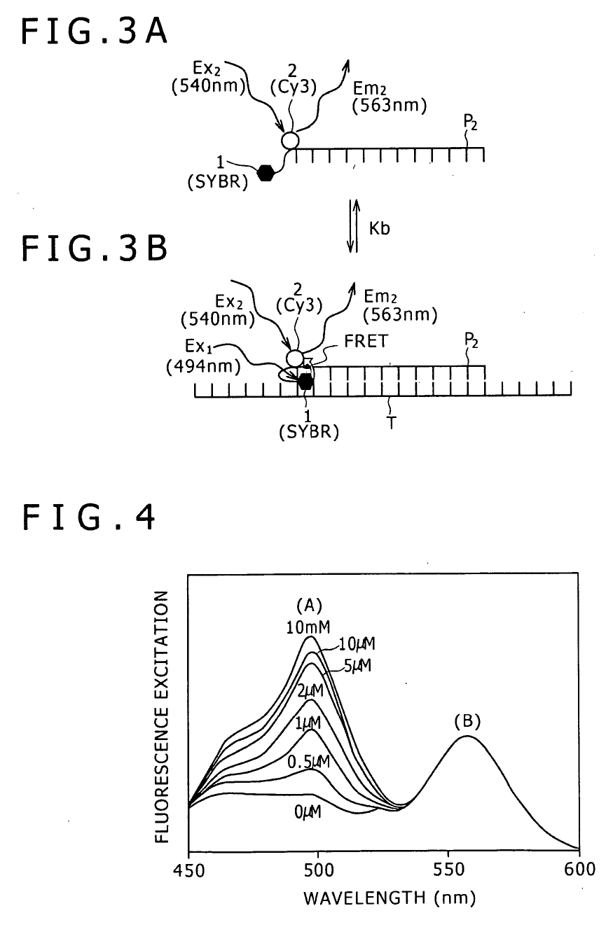

The method of determining the nucleic acid chain T to be detected in real time PCR by using the fluorescence-labeled oligonucleotide P₂ will be explained below with reference to FIGS. 3A and 3B.

The first step is to prepare several kinds of fluorescence-labeled oligonucleotide P₂ each having the base sequence complementary to the nucleic acid chain T to be detected. Each of the fluorescence-labeled oligonucleotide P₂ is labeled with the intercalating fluorescent dye 1 and the non-intercalating fluorescent dye 2 attached to the 5′ end, the former being SYBR Green (having the exciting center wavelength of 494 nm and the emitting center wavelength of 521 nm) and the latter being Cy3 (having the exciting center wavelength of 540 nm and the emitting center wavelength of 563 nm).

Each of the fluorescence-labeled oligonucleotide P₂ is measured for the melting point Tm from its base sequence and the binding constant Kb at a predetermined temperature (e.g., 50° C.). See Formula (3) above.

The binding constant Kb may be obtained from standard samples of known concentration which have the base sequence complementary to each of the fluorescence-labeled oligonucleotide P₂. Specifically, this method includes the following steps. First, standard samples are prepared which are serially diluted to 0 μM, 0.5 μM, 1 μM, 2 μM, 5 μM, 10 μM, and 10 mM. Each standard sample is mixed with the fluorescence-labeled oligonucleotide P₂ and PCR reagent in common use. The standard sample is heated at about 90° C. to denature it into the single-strand. After cooling to a predetermined temperature (e.g., 50° C.), the SYBR Green is excited with excitation ray Ex₁ having a wavelength of 494 nm, and the resulting fluorescence Em₂ having a wavelength of 563 nm is measured for intensity I₁. At the same time, the Cy3 is excited with excitation ray Ex₂ having a wavelength of 540 nm, and the resulting fluorescence Em₂ is measured for intensity I₂. FIG. 4 shows an example of the fluorescence intensity I₁ and I₂ for each concentration. FIG. 3A indicates the fluorescence intensity I₁ for each concentration, and FIG. 3B indicates the fluorescence intensity I₂. By fitting I₁ and I₂ into the formulas (1) and (2) for the least square method, it is possible to calculate the binding constant Kb.

Incidentally, in the formulas (1) and (2), “Conc” denotes the concentration (mol/L) of the standard sample and “Kb” denotes the binding constant of the fluorescence-labeled oligonucleotide P₂. “I₁” denotes the intensity of fluorescence Em₂ (563 nm) due to excitation ray Ex₁ (494 nm), “B₁” denotes its background value (or the intensity of fluorescence Em₂ at 0 μM), “I₂” denotes the intensity of fluorescence Em₂ (563 nm) due to excitation ray Ex₂ (540 nm), and “B₂” denotes its background value. “Ri” denotes the value of R at 0 μM, and “Ra” denotes the value of R at 10 mM.

Out of several kinds of the fluorescence-labeled oligonucleotide P₂ prepared as mentioned above, one which has the binding constant Kb of several μM is selected on the basis of the binding constant Kb calculated from the formula (3) or the formulas (1) and (2).

The fluorescence-labeled oligonucleotide P₂ thus selected is added to the PCR reaction solution of unknown concentration containing the nucleic acid chain T to be detected (such that the final concentration is about 200 μM). The PCR reaction is carried out in the related way by using the fluorescence-labeled oligonucleotide P₂ as the primer.

The annealing temperature of the PCR reaction is the temperature which has been established to calculate the binding constant Kb. (It is 50° C. in the above-mentioned example.) During the annealing reaction, the intensity of fluorescence is detected in the following manner. The result of detection is expressed in terms of the number of cycles in which amplification of the nucleic acid chain T to be detected exponentially proceeds as the result of PCR reaction.

To be concrete, a solution not containing the nucleic acid chain T to be detected is excited with the excitation ray Ex₁ (494 nm) and the intensity B₁ of fluorescence Em₂ (563 nm) is acquired. Then, a solution not containing the nucleic acid chain T to be detected is excited with the excitation ray Ex₂ (540 nm) and the intensity B₂ of fluorescence Em₂ (563 nm) is acquired.

Next, a solution containing the nucleic acid chain T to be detected is excited with the excitation ray Ex₁ (494 nm) and the intensity I₁ of fluorescence Em₂ (563 nm) is acquired. Then, a solution containing the sample is excited with the excitation ray Ex₂ (540 nm) and the intensity I₂ of fluorescence Em₂ (563 nm) is acquired.

The intensity of fluorescence thus obtained is fitted into the formulas (1) and (2) above to calculate the concentration of the nucleic acid chain T (of unknown concentration) to be detected.

As mentioned above, the real time PCR that employs the fluorescence-labeled oligonucleotide P₂ permits one to determine the concentration of the nucleic acid to be detected directly from the fluorescence intensity detected without the necessity of drawing calibration curved at each time of determination. It merely needs to detect the fluorescence intensity I₁ and fluorescence intensity I₂ and fit them into the formulas (1) and (2).

Incidentally, it is not necessary to calculate the binding constant Kb by using the standard samples at each of measurement once it has been obtained. Moreover, it is possible, of course, to calculate the binding constant Kb from the formula (3) above according to the base sequence of the fluorescence-labeled oligonucleolide P2, without using the standard samples.

The advantage of the foregoing method is that the intensity I₁ of fluorescence (which has been calibrated by background) is divided by the intensity I₂ of fluorescence as shown in Formula (2) and this calculation cancels errors due to variation in amount of the intercalating fluorescent dyes and due to instability of light source in the spectrofluorophotometer. Therefore, the foregoing method is free of measurement errors.

The fluorescence-labeled oligonucleotide according to an embodiment of the present invention and the method for acquiring the information about the formation of double-strand of this oligonucleotide can be used to detect the nucleic acid chain and is particularly suitable for determination of nucleic acid chain.

Therefore, the present invention can be applied to elucidate the molecular mechanism of diseases, establish the diagnosis of diseases, and search the target for drug development through the quantitative analysis of the gene expression in cells or tissues during real time PCR.

It should be understood by those skilled in the art that various modifications, combinations, sub-combinations and alterations may occur depending on design requirements and other factors insofar as they are within the scope of the appended claims or the equivalents thereof.

What is claimed is:

1. A fluorescence-labeled oligonucleotide to detect nucleic acid which has a base sequence complementary to the nucleic acid chain to be detected and changes in fluorescence characteristics upon double-strand formation with the nucleic acid chain to be detected.

2. The fluorescence-labeled oligonucleotide as defined in claim 1,
wherein the label is an intercalating fluorescent dye and a non-intercalating fluorescent dye.

3. The fluorescence-labeled oligonucleotide as defined in claim 2, wherein
the intercalating fluorescent dye acquires excited energy upon absorption of light and gives it to the non-intercalating fluorescent dye, and
the non-intercalating fluorescent dye receives the excited energy to emit light.

4. A method for detecting information about fluorescence of the fluorescence-labeled oligonucleotide to detect nucleic acid by using the fluorescence-labeled oligonucleotide to detect nucleic acid,
for acquiring information about double-strand formation between the fluorescence-labeled oligonucleotide to detect nucleic acid and the nucleic acid chain to be detected.

5. The method as defined in claim 4, which is designed to acquire information about the amount of nucleic acid chain to be detected.
